(19) United States
(12) Patent Application Publication
(10) Pub. No.: US 2009/0131343 A1
(43) Pub. Date: May 21, 2009

(54) USE OF BRIDGED MACROLIDES OR TYLOSIN DERIVATIVES IN TREATING INFLAMMATORY BOWEL DISEASES

(76) Inventors: Ly Tam Phan, Quincy, MA (US); Yat Sun Or, Watertown, MA (US)

Correspondence Address: ELMORE PATENT LAW GROUP, PC, 515 Groton Road, Unit 1R, Westford, MA, 01886, US

(21) Appl. No.: 12/270,967

(22) Filed: Nov. 14, 2008

Related U.S. Application Data
(60) Provisional application No. 60/988,257, filed on Nov. 15, 2007.

(51) Int. Cl. A61K 31/7048; A61P 1/00

(52) U.S. Cl. 514/29; 514/450; 514/254.1

(57) ABSTRACT

The invention provides methods utilizing bridged macrolide or tylosin derivatives for the treatment of patients with inflammatory bowel diseases. The methods of the invention provide for the administration to a patient of a therapeutically effective amount of a bridged macrolide or a tylosin derivative, pharmaceutically acceptable derivatives thereof, and combinations thereof for a period of time sufficient to obtain a desired alleviation of one or more symptoms of the inflammatory bowel disease.

### RELATED APPLICATIONS

This application claims the benefit of U.S. provisional application No. 60/988,257 filed on Nov. 15, 2007. The contents of the above applications are incorporated herein by reference.

### TECHNICAL FIELD

The invention provides a method utilizing bridged macrolide or tylosin derivatives for the treatment of patients with inflammatory bowel diseases. The method of the invention provides for the administration to a patient of a therapeutically effective amount of a bridged macrolide or a tylosin derivative, pharmaceutically acceptable derivatives thereof, and combinations thereof for a period of time sufficient to obtain a desired alleviation of one or more symptoms of the inflammatory bowel disease.

### BACKGROUND OF THE INVENTION

Inflammatory bowel diseases (IBD) are defined by chronic, relapsing intestinal inflammation of obscure origin. IBD refers to two distinct disorders, Crohn's disease and ulcerative colitis (IC). Both diseases appear to result from the unrestrained activation of an inflammatory response in the intestine. This inflammatory cascade is thought to be perpetuated through the actions of proinflammatory cytokines and selective activation of lymphocyte subsets. In patients with IBD, ulcers and inflammation of the inner lining of the intestines lead to symptoms of abdominal pain, diarrhea, and rectal bleeding. Ulcerative colitis occurs in the large intestine, while in Crohn's, the disease can involve the entire gastrointestinal (GI) tract as well as the small and large intestines. For most patients, IBD is a chronic condition with symptoms lasting for months to years. It is most common in young adults, but can occur at any age. It is found worldwide, but is most common in industrialized countries.

The clinical symptoms of IBD are intermittent rectal bleeding, crampy abdominal pain, weight loss and diarrhea. Diagnosis of IBD is based on the clinical symptoms and the use of a barium enema, but direct visualization (sigmoidoscopy or colonoscopy) is the most accurate test. Protracted IBD is a risk factor for colon cancer, and treatment of IBD can involve medications and surgery.

IBD affects both children and adults, and has a bimodal age distribution (one peak around 20, and a second around 40). IBD is a chronic, lifelong disease, and is often grouped with other so-called “autoimmune” disorders (e.g. rheumatoid arthritis, type I diabetes mellitus, multiple sclerosis, etc). IBD is found almost exclusively in the industrialized world. The most recent data from the Mayo Clinic suggest an overall incidence of greater than 1 in 100,000 people in the United States, with prevalence data in some studies greater than 1 in 1000. There is a clear trend towards an increasing incidence of IBD in the US and Europe, particularly Crohn's Disease. The basis for this increase is not presently clear. As such, IBD represents the 2^{nd} most common autoimmune disease in the United States (after rheumatoid arthritis).

The most commonly used medications to treat IBD are anti-inflammatory drugs such as the salicylates. The salicylate preparations have been effective in treating mild to moderate disease. They can also decrease the frequency of disease flares when the medications are taken on a prolonged basis. Examples of salicylates include sulfasalazine, olsalazine, mesalamine and azulfidine. All of these medications are given orally in high doses for maximal therapeutic benefit. These medicines are not without side effects. Azulfidine can cause upset stomach when taken in high doses, and rare cases of mild kidney inflammation have been reported with some salicylate preparations. Corticosteroids are more potent and faster-acting than salicylates in the treatment of IBD, but potentially serious side effects limit the use of corticosteroids to patients with more severe disease. Side effects of corticosteroids usually occur with long term use. They include thinning of the bone and skin, infections, diabetes, muscle wasting, rounding of faces, psychiatric disturbances, and, on rare occasions, destruction of hip joints.

In IBD patients that do not respond to salicylates or corticosteroids, medications that suppress the immune system are used. Examples of immunosuppressants include azathioprine and 6-mercaptopurine. Immunosuppressants used in this situation help to control IBD and allow gradual reduction or elimination of corticosteroids. However, immunosuppressants render the patient immuno-compromised and susceptible to many other diseases.

Not withstanding the current therapies to treat IBD, there is a current need for novel, more effective therapies to treat IBD.

### SUMMARY OF THE INVENTION

The present invention provides a method of treating inflammatory bowel disease (IBD) using bridged macrolide system represented by formula (I), (II), (III), (IV) or tylosin derivatives of formula (V) as illustrated below:

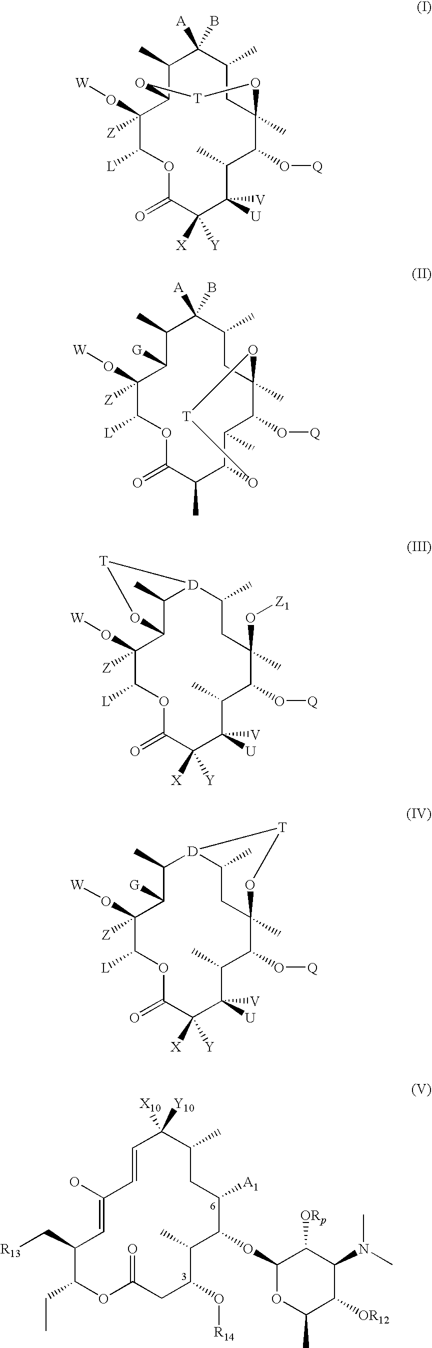

or the racemates, enantiomers, diastereomers, geometric isomers, tautomers, solvates, pharmaceutically acceptable salts, esters and prodrugs thereof,
wherein T is:
(a) —R₁—, where R₁ is substituted or unsubstituted —C₁-C₈ alkylene-, —C₂-C₈ alkenylene- or —C₂-C₈ alkynylene-, containing 0, 1, 2, or 3 heteroatoms selected from O, S or N;
(b) —R₁—(C═O)—R₂—, where R₂ is independently selected from R₁;
(c) —R₁—(C═N-E-R₃)—R₂—, where E is absent, O, NH, NH(CO), NH(CO)NH or NHSO₂ and where R₃ is independently selected from the group consisting of:
   (i) hydrogen;
   (ii) aryl; substituted aryl; heteroaryl; substituted heteroaryl; and
   (iii) —R₄, where R₄ is substituted or unsubstituted —C₁-C₆ alkyl, —C₂-C₆ alkenyl, or —C₂-C₆ alkynyl containing 0, 1, 2, or 3 heteroatoms selected from O, S or N; or
   (iv) —R₅, where R₅ is substituted and unsubstituted —C₃-C₁₂ cycloalkyl containing 0, 1, 2, or 3 heteroatoms selected from O, S or N; and
(d) —R₁—[C(OR₆)(OR₇)]—R₂—, where R₆ and R₇ are selected from the group consisting of C₁-C₁₂ alkyl, aryl or substituted aryl; or R₆ and R₇ taken together is —(CRₐR_{b})ᵣ—, where r is 2 or 3, Rₐ and R_{b} are independently selected from R₃;
(e) —R₁—[C(SR₆)(SR₇)]—R₂—; or
(f) —R₁—(C═CH—R₃)—R₂—;
   one of A and B is hydrogen or hydroxy and the other is selected from:
(a) hydrogen;
(b) —OR₃;
(c) —R₄;
(d) —OC(O)NHR₃;
(e) —OC(O)OR₃;
(f) —NR₈R₉; where R₈ and R₉ are each independently selected from R₃; alternatively, R₈ and R₉ taken together with the nitrogen atom to which they are connected form a 3- to 10-membered ring which may optionally contain one or more heterofunctions selected from the group consisting of: —O—, —NH—, —N(C₁-C₆-alkyl)-, —N(R₁₀)—, —S(O)ₙ—, wherein n=0, 1 or 2, and R₁₀ is selected from aryl; substituted aryl; heteroaryl; and substituted heteroaryl;
(g) —NHC(O)R₃;
(h) —NHS(O)₂R₃;
(i) —NHC(O)OR₃; and
(j) —NHC(O)NHR₃;
   alternatively, A and B taken together with the carbon atom to which they are attached are selected from:
(a) C═O;
(b) C═N-J-R₁₁, where J is absent, O, CO, SO₂, NH, NHC(O), NHC(O)NH or NHSO₂; and wherein R₁₁ is independently selected from halogen and R₃;
(c) C═CH-J-R₁₁;
(d) substituted or unsubstituted, and saturated or unsaturated 5- to 10-membered heterocyclic;

### D is

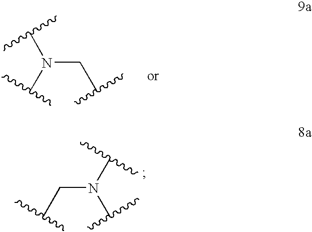

G is selected from the group consisting of:
a) hydrogen;
b) hydroxy;
(c) —O—R₄;
(d) —O—R₁₀.
   Alternatively, G and W taken together to a form cyclic structure selected from:
a)

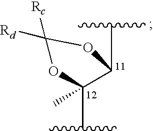

where R_{c} and R_{d} are independent selected from R₃, and
b)

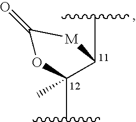

where M is O or N-J-R₂₀, and where J is absent, O, NH, NHC(O), or N═CH; and R₂₀ is selected from the group consisting of:
i. hydrogen;
ii. R₄; and
iii. R₁₀;
   W is selected from:
(a) hydrogen;
(b) —R₄;
(c) —C(O)R₃;
(d) —C(O)O—R₃; and
(e) —C(O)N(R₈R₉);
   when U is hydrogen, V is selected from the group consisting of:
(a) hydrogen;
(b) —OR₃;
(c) —OC(O)R₃;
(d) —OC(O)NHR₃;
(e) —OS(O)₂R₃;
(f) —O-monosaccharide; and
(g) —O-disaccharide;
   alternatively, U and V taken together is oxo;
   L is independently selected from R₄;

### Q is:

(a) —R₃;
(b) —C(O)R₃;
(c) —C(O)NHR₃;
(d) —C(O)OR₃;
(e) —S(O)₂R₃;
(f) monosaccharide;
(g) disaccharide; or
(h) trisaccharide;

### Z is:

(a) hydrogen;
(b) —N₃;
(c) —CN;
(d) —NO₂;
(e) —CONH₂;
(f) —COOH;
(g) —CHO;
(h) —R₄;
(i) —COOR₄;
(j) —C(O)R₄; or
(k) —C(O)NR₈R₉;
   Z₁ is hydrogen or —R₄;
   Each of X and Y is independently:
a) hydrogen;
b) hydroxy;
c) halogen; or
d) —R₄;
   A₁ is selected from the group consisting of:
a) CH₂CHO;
b) CH₂CN;
c) CH₂C═N-J-R₁₁;
d) CH₂E₁R₃₀, where E₁ is absent, O, OC(O), C(O), CONR₃₁, SO₂, CH₂, NR₃₁, R₁, NR₃₁C(O)NR₃₁, NR₃₁SO₂NR₃₂ or NR₃₁SO₂; and wherein R₃₀, R₃₁ and R₃₃ are independently selected from halogen and R₃;
   R₁₄ is selected from the group consisting of:
a) hydrogen;
b) hydroxy protecting group;
c) hydroxy prodrug group;
d) —R₄;
e) —C(O)R₃;
f) —C(O)O—R₃; and
g) —C(O)N(R₈R₉);
   alternatively, A₁ and R₁₄ can be taken together with the atoms to which they are attached to form

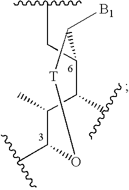

where B₁ is selected from the group consisting of:
a) CHO;
b) CN;
c) HC═N-J-R₁₁;
d) J₁R₃₀, where J₁ is absent, O, OC(O), SO₂, CH₂, NR₃₁, R₁, NR₃₁C(O)NR₃₁, NR₃₁NR₃₂, NR₃₁NR₃₂C(O), NR₃₁SO₂NR₃₂ or NR₃₁SO₂; and wherein R₃₀, R₃₁ and R₃₂ are independently selected from halogen and R₃;
   X₁₀ and Y₁₀ are each independently selected from the group consisting of:
(a) hydrogen;
(b) halogen;
(c) protected hydroxy;
(d) -E-R₃; and
(e) —NR₇R₈;
Alternatively, X₁₀ and Y₁₀ taken together with the carbon atom to which they are attached is:
   (a) C═O;
   (b) C═N—C(O)R₃;
   (c) C═N—OR₉₀, wherein R₉₀ is selected from the group consisting of:
      (1) hydrogen;
      (2) —CH₂O(CH₂)₂OCH₃;
      (3) —CH₂O(CH₂O)ₙCH₃, wherein n is 1, 2, or 3;
      (4) —R₄;
      (5) substituted and unsubstituted, saturated or unsaturated C₃-C₁₂ cycloalkyl;
      (6) substituted and unsubstituted heterocyclic;
      (7) C(O)—(C₃-C₁₂ cycloalkyl);
      (8) C(O)—R₃, wherein R₃ is as previously defined;
      (9) —Si(Rₐ)(R_{b})(R_{c}), wherein Rₐ, R_{b} and R_{c} are each independently selected from the group consisting of C₁-C₁₂ alkyl, aryl and substituted aryl; or
      (10) C(R₉₀)(R₁₀₀)—O—R₁₁₀, wherein R₉₀ and R₁₀₀ taken together with the carbon atom to which they are attached form a C₃ to C₁₂ cycloalkyl group or each independently is selected from the group consisting of: hydrogen and C₁-C₁₂ alkyl; and R₁₁₀ is selected from the group consisting of:
         (i) —R₄;
         (ii) substituted and unsubstituted, saturated or unsaturated —C₃-C₁₂ cycloalkyl; and
         (iii) —Si(Rₐ)(R_{b})(R_{c}), wherein Rₐ, R_{b} and R_{c} are as previously defined;

### R₁₂ is -M₁-Q₁,

where M₁ is:
(a) absent;
(b) —C(O)—;
(c) —C(O)N(R₃)—; or
(d) —R₁—;

and where Q₁ is:
(a) hydrogen;
(b) hydroxy protecting group;
(c) hydroxy prodrug group;
(d)

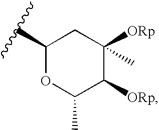

where Rp is hydrogen, a hydroxy protecting group or a hydroxy prodrug group;
(e) —R₃;
(f) —OR₃;
(g) —NR₇R₈; or
(h) substituted or unsubstituted heterocyclic;
   R₁₃ is -G₁-M₁-W₁, where G₁ is absent, —O—, or —N(R₃)—, and where W₁ is:
(a) hydrogen;
(b) hydroxy protecting group;
(c) hydroxy prodrug group
(d) halogen;
(e)

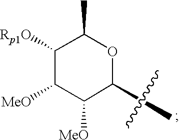

(f) —R₃;
(g) —OR₃; or
(h) substituted or unsubstituted heterocyclic;
   Rₚ and Rₚ₁ are independently hydrogen, a hydroxy protecting group or a hydroxy prodrug group.

### DETAILED DESCRIPTION OF THE INVENTION

In a first embodiment is a method for treating IBD by administering to a patient in need of compounds represented by formula I, II, III, IV or V as illustrated above, or a pharmaceutically acceptable salt, ester or prodrug thereof.

In one embodiment is a method for treating IBD by administering to a patient in need of compounds represented by formula VI or a pharmaceutically acceptable salt, ester or prodrug thereof:

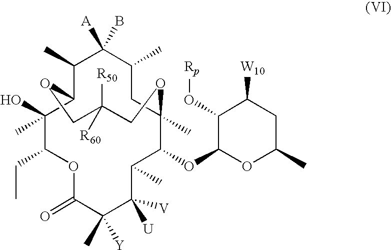

where R₅₀ and R₆₀ are independently selected from the group consisting of:
a) hydrogen;
b) deuterium;
c) hydroxy;
d) activated hydroxy;
e) N₃;
f) NH₂;
g) CN;
h) protected hydroxy;
i) protected amino;
j) -L₁-R₃, where L₁ is absent, O, OC(O), S, S(O), SO₂, NH, NCH₃, NHC(O), NHC(O)NH or NHSO₂; and
k) substituted or unsubstituted heterocyclic;
   alternatively, R₅₀ and R₆₀ can be taken together with the carbon atom to which they are attached is selected from the group consisting of:
a) C═O;
b) C(OR₆)(OR₇);
c) C(SR₆)(SR₇);
d) C═CHR₃;
e) C═NRₐₚ; where Rₐₚ is amino protecting group; and
f) C═N-E-R₃;
   W₁₀ is —NR₈R₉; and A, B, U, V, Y, R₃, R₆, R₇, R₈, R₉ and Rₚ are as previously defined.

In one embodiment is a method for treating IBD by administering to a patient in need of compounds represented by formula VII or a pharmaceutically acceptable salt, ester or prodrug thereof:

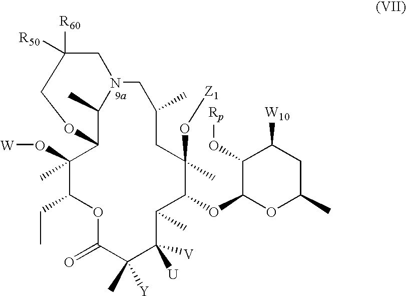

where R₅₀, R₆₀, U, V, Y, W, W₁₀, Z₁ and Rₚ are as previously defined.

In one embodiment is a method for treating IBD by administering to a patient in need of compounds represented by formula VIII or a pharmaceutically acceptable salt, ester or prodrug thereof:

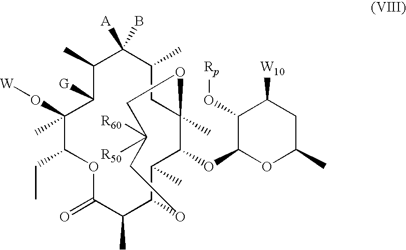

where R₅₀, R₆₀, A, B, G, W, W₁₀ and Rₚ are as previously defined.

In one embodiment is a method for treating IBD by administering to a patient in need of compounds represented by formula IX or a pharmaceutically acceptable salt, ester or prodrug thereof:

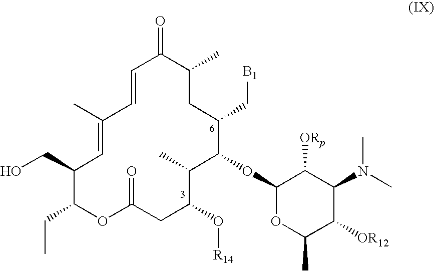

where B₁, R₁₂, R₁₄ and Rₚ are as previously defined.

Representative compounds that can be used for treating IBD according to the invention are those selected from the group consisting of:

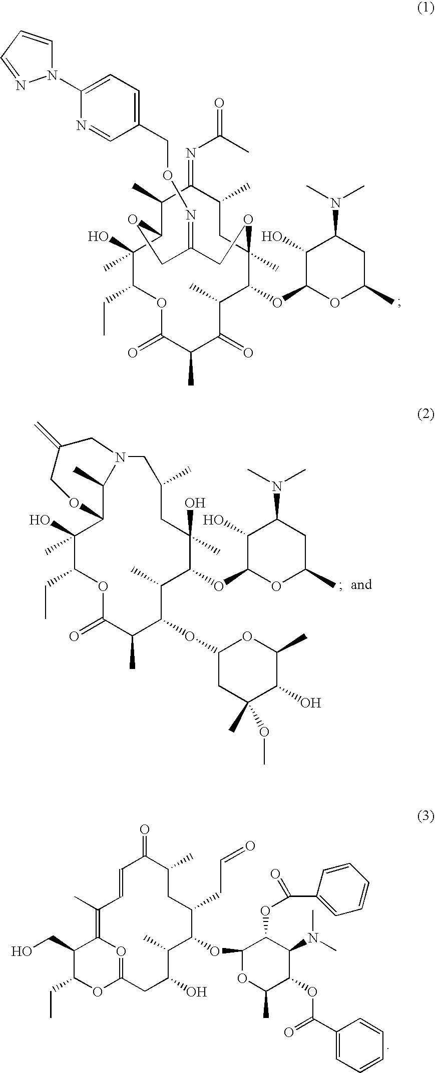

A further embodiment of the present invention includes pharmaceutical compositions comprising any single compound delineated herein, or a pharmaceutically acceptable salt, ester, or prodrug thereof, with a pharmaceutically acceptable carrier or excipient.

Yet another embodiment of the present invention is a pharmaceutical composition comprising a combination of two or more compounds delineated herein, or a pharmaceutically acceptable salt, ester, or prodrug thereof, with a pharmaceutically acceptable carrier or excipient.

Yet a further embodiment of the present invention is a pharmaceutical composition comprising any single compound delineated herein in combination with one or more antibiotics known in the art (such as penicillin, amoxicillin, azithromycin, erythromycin, ciprofloxacin, telithromycin, cethromycin, and the like), or a pharmaceutically acceptable salt, ester, or prodrug thereof, with a pharmaceutically acceptable carrier or excipient.

In one embodiment, a compound of the present invention can be used in combination with other drugs used in the treatment of inflammatory bowel disease. For example, compounds of the invention can be used in combination with drugs such as, but not limited to, auranofin, azathioprine, cyclophosphamide, cyclosporine, etanercept, hydroxychloroquine, inflaximab, leflunomide, methotrexate, minocycline, mycophenalate mofetil, penicillamine, sulfasalazine, tacrolimus, and the like.

In one embodiment, a compound of the invention may be used to treat an inflammatory disease including, but not exclusive to, autoimmune diseases involving multiple organs, such as systemic lupus erythematosus (SLE) and scleroderma, specific tissues or organs such as the musculoskeletal tissue (rheumatoid arthritis and ankylosing spondylitis), gastro-intestinal tract (Crohn's disease and ulcerative colitis), the central nervous system (Alzheimer's, multiple sclerosis, motor neurone disease, Parkinson's disease and chronic fatigue syndrome), pancreatic beta cells (insulin-dependent diabetes mellitus), the adrenal gland (Addison's disease), the kidney (Goodpasture's syndrome, IgA nephropathy and interstitial nephritis), exocrine glands (Sjogren's syndrome and autoimmune pancreatitis) and skin (psoriasis and atopic dermatitis), chronic inflammatory diseases such as osteoarthritis, periodontal disease, diabetic nephropathy, chronic obstructive pulmonary disease, atherosclerosis, graft versus host disease, chronic pelvic inflammatory disease, endometriosis, chronic hepatitis and tuberculosis and IgE-mediated (Type I) hypersensitivities such as rhinitis, asthma, anaphylaxis and dermatitis. Dermatitis conditions that may be treated include actinic keratosis, acne rosacea, acne vulgaris, allergic contact dermatitis, angioedema, atopic dermatitis, bullous pemiphigoid, cutaneous drug reactions, erythema multiforme, lupus erythrometosus, photodermatitis, psoriasis, psoriatic arthritis, scleroderma and urticaria.

This invention also relates to the treatment of subjects (including man and/or mammalian animals raised in the dairy, meat or fur industries or as pets) suffering from chronic, acute or neuropathic pain. Compounds of the invention, and in particular, the preferred enantiomers or diastereomers of compounds of the invention, can be used among other things in the treatment of pain conditions such as acute and chronic pain (as well as, but not limited to, pain associated with cancer, surgery, arthritis, dental surgery, trauma, musculo-skeletal injury or disease and visceral diseases) and migraine headache. Painful conditions that can be treated also include neuropathic pain (post-herpetic neuralgia, diabetic neuropathy, drug induced neuropathy, HIV mediated neuropathy, sympathetic reflex dystrophy or causalgia, fibromyalgia, myofacial pain, entrapment neuropathy, phantom limb pain, trigeminal neuralgia. Neuropathic conditions include central pain related to stroke, multiple sclerosis, spinal cord injury, arachnoiditis, neoplasms, syringomyelia, Parkinson's and epilepsia.

It will often be advantageous to use compounds of the invention in combination with another drug used for pain therapy. Such another drug may be an opiate or a non-opiate such as baclofen. Especially for the treatment of neuropathic pain, coadministration with gabapentin is preferred. Other compounds that may be used include acetaminophen, a non-steroidal anti-inflammatory drug, a narcotic analgesic, a local anaesthetic, an NMDA antagonist, a neuroleptic agent, an anti-convulsant, an anti-spasmodic, an anti-depressant or a muscle relaxant.

Compounds may be used according to the invention when the patient is also administered or in combination with another therapeutic agent selected from corticosteroids (examples include cortisol, cortisone, hydrocortisone, dihydrocortisone, fludrocortisone, prednisone, prednisolone, deflazacort, flunisolide, beconase, methylprednisolone, triamcinolone, betamethasone, and dexamethasone), disease modifying anti-rheumatic drugs (DMARDs) (examples include azulfidine, aurothiomalate, bucillamine, chlorambucil, cyclophosphamide, leflunomide, methotrexate, mizoribine, penicillamine and sulphasalazine), immunosuppressants (examples include azathioprine, cyclosporin, mycophenolate), COX inhibitors (examples include aceclofenac, acemetacin, alcofenac, alminoprofen, aloxipirin, amfenac, aminophenazone, antraphenine, aspirin, azapropazone, benorilate, benoxaprofen, benzydamine, butibufen, celecoxib, chlorthenoxacine, choline salicylate, chlometacin, dexketoprofen, diclofenac, diflunisal, emorfazone, epirizole, etodolac, feclobuzone, felbinac, fenbufen, fenclofenac, flurbiprofen, glafenine, hydroxyethyl salicylate, ibuprofen, indomethacin, indoprofen, ketoprofen, ketorolac, lactyl phenetidin, loxoprofen, mefenamic acid, metamizole, mofebutazone, mofezolac, nabumetone, naproxen, nifenazone, oxametacin, phenacetin, pipebuzone, pranoprofen, propyphenazone, proquazone, rofecoxib, salicylamide, salsalate, sulindac, suprofen, tiaramide, tinoridine, tolfenamic acid, zomepirac), neutralising antibodies (examples include etanercept and infliximab) and antibiotics (examples include doxycycline and minocycline).

### DEFINITIONS

Listed below are definitions of various terms used to describe this invention. These definitions apply to the terms as they are used throughout this specification and claims, unless otherwise limited in specific instances, either individually or as part of a larger group.

The phrase “symptoms of IBD” is herein defined as detected symptoms such as abdominal pain, diarrhea, rectal bleeding, weight loss, fever, loss of appetite, and other more serious complications, such as dehydration, anemia and malnutrition. A number of such symptoms are subject to quantitative analysis (e.g., weight loss, fever, anemia, etc.). Some symptoms are readily determined from a blood test (e.g. anemia) or a test that detects the presence of blood (e.g., rectal bleeding). The phrase “wherein said symptoms are reduced” refers to a qualitative or quantitative reduction in detectable symptoms, including but not limited to a detectable impact on the rate of recovery from disease (e.g., rate of weight gain).

The term “aryl,” as used herein, refers to a mono- or polycyclic carbocyclic ring system including, but not limited to, phenyl, naphthyl, tetrahydronaphthyl, indanyl, idenyl.

The term “heteroaryl,” as used herein, refers to a mono- or polycyclic aromatic radical having one or more ring atom selected from S, O and N; and the remaining ring atoms are carbon, wherein any N or S contained within the ring may be optionally oxidized. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl.

In accordance with the invention, any of the aryls, substituted aryls, heteroaryls and substituted heteroaryls described herein, can be any aromatic group. Aromatic groups can be substituted or unsubstituted.

The terms “C₁-C₈ alkyl,” or “C₁-C₁₂ alkyl,” as used herein, refer to saturated, straight- or branched-chain hydrocarbon radicals containing between one and eight, or one and twelve carbon atoms, respectively. Examples of C₁-C₈ alkyl radicals include, but are not limited to, methyl, ethyl, propyl, isopropyl, n-butyl, tert-butyl, neopentyl, n-hexyl, heptyl and octyl radicals; and examples of C₁-C₁₂ alkyl radicals include, but are not limited to, ethyl, propyl, isopropyl, n-hexyl, octyl, decyl, dodecyl radicals.

The term “C₂-C₈ alkenyl,” as used herein, refer to straight- or branched-chain hydrocarbon radicals containing from two to eight carbon atoms having at least one carbon-carbon double bond by the removal of a single hydrogen atom. Alkenyl groups include, but are not limited to, for example, ethenyl, propenyl, butenyl, 1-methyl-2-buten-1-yl, heptenyl, octenyl, and the like.

The term “C₂-C₈ alkynyl,” as used herein, refer to straight- or branched-chain hydrocarbon radicals containing from two to eight carbon atoms having at least one carbon-carbon triple bond by the removal of a single hydrogen atom. Representative alkynyl groups include, but are not limited to, for example, ethynyl, 1-propynyl, 1-butynyl, heptynyl, octynyl, and the like.

The term “C₃-C₈-cycloalkyl”, or “C₃-C₁₂-cycloalkyl,” as used herein, refers to a monocyclic or polycyclic saturated carbocyclic ring compound. Examples of C₃-C₈-cycloalkyl include, but not limited to, cyclopropyl, cyclobutyl, cyclopentyl, cyclohexyl, cyclopentyl and cyclooctyl; and examples of C₃-C₁₂-cycloalkyl include, but not limited to, cyclopropyl, cyclobutyl, cyclopentyl, cyclohexyl, bicyclo[2.2.1]heptyl, and bicyclo[2.2.2]octyl.

The term “C₃-C₈ cycloalkenyl”, or “C₃-C₁₂ cycloalkenyl” as used herein, refers to monocyclic or polycyclic carbocyclic ring compound having at least one carbon-carbon double bond. Examples of C₃-C₈ cycloalkenyl include, but not limited to, cyclopropenyl, cyclobutenyl, cyclopentenyl, cyclohexenyl, cycloheptenyl, cyclooctenyl, and the like; and examples of C₃-C₁₂ cycloalkenyl include, but not limited to, cyclopropenyl, cyclobutenyl, cyclopentenyl, cyclohexenyl, cycloheptenyl, cyclooctenyl, and the like.

It is understood that any alkyl, alkenyl, alkynyl and cycloalkyl moiety described herein can also be an aliphatic group, an alicyclic group or a heterocyclic group. An “aliphatic” group is a non-aromatic moiety that may contain any combination of carbon atoms, hydrogen atoms, halogen atoms, oxygen, nitrogen or other atoms, and optionally contain one or more units of unsaturation, e.g., double and/or triple bonds. An aliphatic group may be straight chained, branched or cyclic and preferably contains between about 1 and about 24 carbon atoms, more typically between about 1 and about 12 carbon atoms. In addition to aliphatic hydrocarbon groups, aliphatic groups include, for example, polyalkoxyalkyls, such as polyalkylene glycols, polyamines, and polyimines, for example. Such aliphatic groups may be further substituted.

The term “alicyclic,” as used herein, denotes a monovalent group derived from a monocyclic or bicyclic saturated carbocyclic ring compound by the removal of a single hydrogen atom. Examples include, but not limited to, cyclopropyl, cyclobutyl, cyclopentyl, cyclohexyl, bicyclo[2.2.1]heptyl, and bicyclo[2.2.2]octyl. Such alicyclic groups may be further substituted.

The terms “heterocyclic” or “heterocycloalkyl” can be used interchangeably and referred to a non-aromatic ring or a bi- or tri-cyclic group fused system, where (i) each ring system contains at least one heteroatom independently selected from oxygen, sulfur and nitrogen, (ii) each ring system can be saturated or unsaturated (iii) the nitrogen and sulfur heteroatoms may optionally be oxidized, (iv) the nitrogen heteroatom may optionally be quaternized, (iv) any of the above rings may be fused to an aromatic ring, and (v) the remaining ring atoms are carbon atoms which may be optionally oxo-substituted. Representative heterocyclic groups include, but are not limited to, 1,3-dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, and tetrahydrofuryl. Such heterocyclic groups may be further substituted.

The term “substituted” refers to substitution by independent replacement of one, two, or three or more of the hydrogen atoms thereon with substituents including, but not limited to, —F, —Cl, —Br, —I, —OH, protected hydroxy, —NO₂, —CN, —NH₂, protected amino, oxo, thioxo, —NH—C₁-C₁₂-alkyl, —NH—C₂-C₈-alkenyl, —NH—C₂-C₈-alkynyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₈-alkenyl, —O—C₂-C₈-alkynyl, —O—C₃-C₁₂-cycloalkyl, —O-aryl, —O-heteroaryl, —O-heterocycloalkyl, —C(O)—C₁-C₁₂-alkyl, —C(O)—C₂-C₈-alkenyl, —C(O)—C₂-C₈-alkynyl, —C(O)—C₃-C₁₂-cycloalkyl, —C(O)-aryl, —C(O)-heteroaryl, —C(O)-heterocycloalkyl, —CONH₂, —CONH—C₁-C₁₂-alkyl, —CONH—C₂-C₈-alkenyl, —CONH—C₂-C₈-alkynyl, —CONH—C₃-C₁₂-cycloalkyl, —CONH-aryl, —CONH-heteroaryl, —CONH-heterocycloalkyl, —OCO₂—C₁-C₁₂-alkyl, —OCO₂—C₂-C₈-alkenyl, —OCO₂—C₂-C₈-alkynyl, —OCO₂—C₃-C₁₂-cycloalkyl, —OCO₂-aryl, —OCO₂-heteroaryl, —OCO₂-heterocycloalkyl, —OCONH₂, —OCONH—C₁-C₁₂-alkyl, —OCONH—C₂-C₈-alkenyl, —OCONH—C₂-C₈-alkynyl, —OCONH—C₃-C₁₂-cycloalkyl, —OCONH-aryl, —OCONH-heteroaryl, —OCONH— heterocycloalkyl, —NHC(O)—C₁-C₁₂-alkyl, —NHC(O)—C₂-C₈-alkenyl, —NHC(O)—C₂-C₈-alkynyl, —NHC(O)—C₃-C₁₂-cycloalkyl, —NHC(O)-aryl, —NHC(O)-heteroaryl, —NHC(O)-heterocycloalkyl, —NHCO₂—C₁-C₁₂-alkyl, —NHCO₂—C₂-C₈-alkenyl, —NHCO₂—C₂-C₈-alkynyl, —NHCO₂—C₃-C₁₂-cycloalkyl, —NHCO₂-aryl, —NHCO₂-heteroaryl, —NHCO₂— heterocycloalkyl, —NHC(O)NH₂, —NHC(O)NH—C₁-C₁₂-alkyl, —NHC(O)NH—C₂-C₈-alkenyl, —NHC(O)NH—C₂-C₈-alkynyl, —NHC(O)NH—C₃-C₁₂-cycloalkyl, —NHC(O)NH-aryl, —NHC(O)NH-heteroaryl, —NHC(O)NH-heterocycloalkyl, NHC(S)NH₂, —NHC(S)NH—C₁-C₁₂-alkyl, —NHC(S)NH—C₂-C₈-alkenyl, —NHC(S)NH—C₂-C₈-alkynyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, —NHC(NH)NH—C₁-C₁₂-alkyl, —NHC(NH)NH—C₂-C₈-alkenyl, —NHC(NH)NH—C₂-C₈-alkynyl, —NHC(NH)NH—C₃-C₁₂-cycloalkyl, —NHC(NH)NH-aryl, —NHC(NH)NH-heteroaryl, —NHC(NH)NH-heterocycloalkyl, —NHC(NH)—C₁-C₁₂-alkyl, —NHC(NH)—C₂-C₈-alkenyl, —NHC(NH)—C₂-C₈-alkynyl, —NHC(NH)—C₃-C₁₂-cycloalkyl, —NHC(NH)-aryl, —NHC(NH)-heteroaryl, —NHC(NH)-heterocycloalkyl, —C(NH)NH—C₁-C₁₂-alkyl, —C(NH)NH—C₂-C₈-alkenyl, —C(NH)NH—C₂-C₈-alkynyl, —C(NH)NH—C₃-C₁₂-cycloalkyl, —C(NH)NH-aryl, —C(NH)NH-heteroaryl, —C(NH)NH-heterocycloalkyl, —S(O)—C₁-C₁₂-alkyl, —S(O)—C₂-C₈-alkenyl, —S(O)—C₂-C₈-alkynyl, —S(O)—C₃-C₁₂-cycloalkyl, —S(O)-aryl, —S(O)-heteroaryl, —S(O)-heterocycloalkyl —SO₂NH₂, —SO₂NH—C₁-C₁₂-alkyl, —SO₂NH—C₂-C₈-alkenyl, —SO₂NH—C₂-C₈-alkynyl, —SO₂NH—C₃-C₁₂-cycloalkyl, —SO₂NH-aryl, —SO₂NH-heteroaryl, —SO₂NH-heterocycloalkyl, —NHSO₂—C₁-C₁₂-alkyl, —NHSO₂—C₂-C₈-alkenyl, —NHSO₂—C₂-C₈-alkynyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₈-alkenyl, —S—C₂-C₈-alkynyl, —S—C₃-C₁₂-cycloalkyl, —S-aryl, —S-heteroaryl, —S-heterocycloalkyl, or methylthiomethyl. It is understood that the aryls, heteroaryls, alkyls, and the like can be further substituted.

The term “monosaccharide” embraces radicals of cladinose, allose, altrose, arabinose, erythrose, erythrulose, fructose, D-fucitol, L-fucitol, fucosamine, fucose, galactosamine, D-galactosaminitol, galactose, glucosamine, glucosaminitol, glucose, glyceraldehyde, glycerol, glycerone, gulose, idose, lyxose, mannosamine, annose, psicose, quinovose, quinovosamine, rhamnitol, rhamnosamine, rhamnose, ribose, ribulose, sorbose, tagatose, tartaric acid, threose, xylose, xylulose and the like. The monosaccharide may further be a deoxy sugar (alcoholic hydroxy group replaced by hydrogen), amino sugar (alcoholic hydroxy group replaced by amino group), a thio sugar (alcoholic hydroxy group replaced by thiol, or C═O replaced by C═S, or a ring oxygen of cyclic form replaced by sulfur), a seleno sugar, a telluro sugar, an aza sugar (ring carbon replaced by nitrogen), an imino sugar (ring oxygen replaced by nitrogen), a phosphano sugar (ring oxygen replaced with phosphorus), a phospha sugar (ring carbon replaced with phosphorus), a C-substituted monosaccharide (hydrogen at a non-terminal carbon atom replaced with carbon), an unsaturated monosaccharide, an alditol (carbonyl group replaced with CHOH group), aldonic acid (aldehydic group replaced by carboxy group), a ketoaldonic acid, a uronic acid, an aldaric acid, and so forth. Amino sugars include amino monosaccharides, preferably galactosamine, glucosamine, mannosamine, fucosamine, quinovosamine, neuraminic acid, muramic acid, lactosediamine, acosamine, bacillosamine, daunosamine, desosamine, forosamine, garosamine, kanosamine, kansosamine, mycaminose, mycosamine, perosamine, pneumosamine, purpurosamine, rhodosamine. It is understood that the monosaccharide and the like can be further substituted.

The terms “disaccharide”, “trisaccharide” and “polysaccharide” embrace radicals of abequose, amicetose, amylose, apiose, arcanose, ascarylose, ascorbic acid, boivinose, cellobiose, cellotriose, chacotriose, chalcose, colitose, cymarose, 2-deoxyribose, 2-deoxyglucose, diginose, digitalose, digitoxose, evalose, evemitrose, gentianose, gentiobiose, hamamelose, inulin, isolevoglucosenone, isomaltose, isomaltotriose, isopanose, kojibiose, lactose, lactosamine, lactosediamine, laminarabiose, levoglucosan, levoglucosenone, β-maltose, manninotriose, melezitose, melibiose, muramic acid, mycarose, mycinose, neuraminic acid, nigerose, nojirimycin, noviose, oleandrose, panose, paratose, planteose, primeverose, raffinose, rhodinose, rutinose, sarmentose, sedoheptulose, sedoheptulosan, solatriose, sophorose, stachyose, streptose, sucrose, α,α-trehalose, trehalosamine, turanose, tyvelose, umbelliferose and the like. Further, it is understood that the “disaccharide”, “trisaccharide” and “polysaccharide” and the like can be further substituted. Disaccharide also includes amino sugars and their derivatives, particularly, a mycaminose derivatized at the C-4′ position or a 4 deoxy-3-amino-glucose derivatized at the C-6′ position.

The term “trisaccharide” includes amino sugars and halo sugars, where halo sugars is saccharide group having at least one halogen substituent.

The term “halogen,” as used herein, refers to an atom selected from fluorine, chlorine, bromine and iodine.

The term “hydroxy activating group”, as used herein, refers to a labile chemical moiety which is known in the art to activate a hydroxy group so that it will depart during synthetic procedures such as in a substitution or an elimination reaction. Examples of hydroxy activating group include, but not limited to, mesylate, tosylate, triflate, p-nitrobenzoate, phosphonate and the like.

The term “activated hydroxy”, as used herein, refers to a hydroxy group activated with a hydroxy activating group, as defined above, including mesylate, tosylate, triflate, p-nitrobenzoate, phosphonate groups, for example.

The term “hydroxy protecting group,” as used herein, refers to a labile chemical moiety which is known in the art to protect a hydroxy group against undesired reactions during synthetic procedures. After said synthetic procedure(s) the hydroxy protecting group as described herein may be selectively removed. Hydroxy protecting groups as known in the art are described generally in T. H. Greene and P. G. M. Wuts, Protective Groups in Organic Synthesis, 3rd edition, John Wiley & Sons, New York (1999). Examples of hydroxy protecting groups include benzyloxycarbonyl, 4-nitrobenzyloxycarbonyl, 4-bromobenzyloxycarbonyl, 4-methoxybenzyloxycarbonyl, methoxycarbonyl, tert-butoxycarbonyl, isopropoxycarbonyl, diphenylmethoxycarbonyl, 2,2,2-trichloroethoxycarbonyl, 2-(trimethylsilyl)ethoxycarbonyl, 2-furfuryloxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, 1,1-dimethyl-2-propenyl, 3-methyl-3-butenyl, allyl, benzyl, para-methoxybenzyldiphenylmethyl, triphenylmethyl(trityl), tetrahydrofuryl, methoxymethyl, methylthiomethyl, benzyloxymethyl, 2,2,2-triehloroethoxymethyl, 2-(trimethylsilyl)ethoxymethyl, methanesulfonyl, para-toluenesulfonyl, trimethylsilyl, triethylsilyl, triisopropylsilyl, and the like. Preferred hydroxy protecting groups for the present invention are acetyl (Ac or —C(O)CH₃), benzoyl (Bz or —C(O)C₆H₅), and trimethylsilyl (TMS or —Si(CH₃)₃).

The term “protected hydroxy,” as used herein, refers to a hydroxy group protected with a hydroxy protecting group, as defined above, including benzoyl, acetyl, trimethylsilyl, triethylsilyl, methoxymethyl groups, for example.

The term “hydroxy prodrug group”, as used herein, refers to a promoiety group which is known in the art to change the physicochemical, and hence the biological properties of a parent drug in a transient manner by covering or masking the hydroxy group. After said synthetic procedure(s), the hydroxy prodrug group as described herein must be capable of reverting back to hydroxy group in vivo. Hydroxy prodrug groups as known in the art are described generally in Kenneth B. Sloan, Prodrugs, Topical and Ocular Drug Delivery, (Drugs and the Pharmaceutical Sciences; Volume 53), Marcel Dekker, Inc., New York (1992).

The term “amino protecting group,” as used herein, refers to a labile chemical moiety which is known in the art to protect an amino group against undesired reactions during synthetic procedures. After said synthetic procedure(s) the amino protecting group as described herein may be selectively removed. Amino protecting groups as known in the art are described generally in T. H. Greene and P. G. M. Wuts, Protective Groups in Organic Synthesis, 3rd edition, John Wiley & Sons, New York (1999). Examples of amino protecting groups include, but are not limited to, t-butoxycarbonyl, 9-fluorenylmethoxycarbonyl, benzyloxycarbonyl, and the like.

The term “leaving group” means a functional group or atom which can be displaced by another functional group or atom in a substitution reaction, such as a nucleophilic substitution reaction. By way of example, representative leaving groups include chloro, bromo and iodo groups; sulfonic ester groups, such as mesylate, tosylate, brosylate, nosylate and the like; and acyloxy groups, such as acetoxy, trifluoroacetoxy and the like.

The term “protected amino,” as used herein, refers to an amino group protected with an amino protecting group as defined above.

The term “aprotic solvent,” as used herein, refers to a solvent that is relatively inert to proton activity, i.e., not acting as a proton-donor. Examples include, but are not limited to, hydrocarbons, such as hexane and toluene, for example, halogenated hydrocarbons, such as, for example, methylene chloride, ethylene chloride, chloroform, and the like, heterocyclic compounds, such as, for example, tetrahydrofuran and N-methylpyrrolidinone, and ethers such as diethyl ether, bis-methoxymethyl ether. Such compounds are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of aprotic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, NY, 1986.

The term “protic solvent’ as used herein, refers to a solvent that tends to provide protons, such as an alcohol, for example, methanol, ethanol, propanol, isopropanol, butanol, t-butanol, and the like. Such solvents are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of protogenic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, NY, 1986.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The synthesized compounds can be separated from a reaction mixture and further purified by a method such as column chromatography, high pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The term “subject” as used herein refers to an animal. Preferably the animal is a mammal. More preferably the mammal is a human. A subject also refers to, for example, dogs, cats, horses, cows, pigs, guinea pigs, fish, birds and the like.

The compounds of this invention may be modified by appending appropriate functionalities to enhance selective biological properties. Such modifications are known in the art and may include those which increase biological penetration into a given biological system (e.g., blood, lymphatic system, central nervous system), increase oral availability, increase solubility to allow administration by injection, alter metabolism and alter rate of excretion.

The compounds described herein contain one or more asymmetric centers and thus give rise to enantiomers, diastereomers, and other stereoisomeric forms that may be defined, in terms of absolute stereochemistry, as (R)— or (S)—, or as (D)- or (L)- for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers or cis- and trans-isomers. Likewise, all tautomeric forms are also intended to be included. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid. Examples of pharmaceutically acceptable include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present invention. “Prodrug”, as used herein means a compound which is convertible in vivo by metabolic means (e.g. by hydrolysis) to a compound of the invention. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 113-191 (1991); Bundgaard, et al., Journal of Drug Deliver Reviews, 8:1-38 (1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

The present invention also relates to solvates of the compounds of the invention, for example hydrates.

This invention also encompasses pharmaceutical compositions containing, and methods of treating bacterial infections through administering, pharmaceutically acceptable prodrugs of compounds of the invention. For example, compounds of the invention having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the invention. The amino acid residues include but are not limited to the 20 naturally occurring amino acids commonly designated by three letter symbols and also includes 4-hydroxyproline, hydroxyysine, demosine, isodemosine, 3-methylhistidine, norvalin, beta-alanine, gamma-aminobutyric acid, citrulline, homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

### Pharmaceutical Compositions

The pharmaceutical compositions of the present invention comprise a therapeutically effective amount of a compound of the present invention formulated together with one or more pharmaceutically acceptable carriers or excipients.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminun hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition of the invention is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics, particularly aerosolized antibiotics, is known in the art (see, for example U.S. Pat. No. 5,767,068 to VanDevanter et al., U.S. Pat. No. 5,508,269 to Smith et al., and WO 98/43,650 by Montgomery, all of which are incorporated herein by reference). A discussion of pulmonary delivery of antibiotics is also found in U.S. Pat. No. 6,014,969, incorporated herein by reference.

According to the methods of treatment of the present invention, bacterial infections, cystic fibrosis and inflammatory conditions are treated or prevented in a patient such as a human or another animal by administering to the patient a therapeutically effective amount of a compound of the invention, in such amounts and for such time as is necessary to achieve the desired result.

By a “therapeutically effective amount” of a compound of the invention is meant an amount of the compound which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the compound described above may range from about 0.1 mg/Kg to about 500 mg/Kg, preferably from about 1 to about 50 mg/Kg. Effective doses will also vary depending on route of administration, as well as the possibility of co-usage with other agents. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

The total daily dose of the compounds of this invention administered to a human or other animal in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In general, treatment regimens according to the present invention comprise administration to a patient in need of such treatment from about 10 mg to about 1000 mg of the compound(s) of this invention per day in single or multiple doses.

The compounds of the formulae described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.1 to about 500 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with pharmaceutically excipients or carriers to produce a single dosage form will vary depending upon the host treated and the particular mode of administration. A typical preparation will contain from about 5% to about 95% active compound (w/w). Alternatively, such preparations may contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

When the compositions of this invention comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

Unless otherwise defined, all technical and scientific terms used herein are accorded the meaning commonly known to one of ordinary skill in the art. All publications, patents, published patent applications, and other references mentioned herein are hereby incorporated by reference in their entirety.

### Abbreviations

Abbreviations which may appear in the following synthetic schemes and examples are:
Ac for acetyl;
AcOH for acetic acid;
AIBN for azobisisobutyronitrile;
BINAP for 2,2′-bis(diphenylphosphino)-1,1′-binaphthyl;
Boc₂O for di-tert-butyl-dicarbonate;
Boc for t-butoxycarbonyl;
Bpoc for 1-methyl-1-(4-biphenylyl)ethyl carbonyl;
Bz for benzoyl;
Bn for benzyl;
BocNHOH for tert-butyl N-hydroxycarbamate;
t-BuOK for potassium tert-butoxide;
Bu₃SnH for tributyltin hydride;
BOP for (benzotriazol-1-yloxy)tris(dimethylamino)phosphonium Hexafluorophosphate;
Brine for sodium chloride solution in water;
CDI for carbonyldiimidazole;
CH₂Cl₂ for dichloromethane;
CH₃ for methyl;
CH₃CN for acetonitrile;
Cs₂CO₃ for cesium carbonate;
CuCl for copper (I) chloride;
CuI for copper (I) iodide;
dba for dibenzylidene acetone;
dppb for diphenylphosphino butane;
DBU for 1,8-diazabicyclo[5.4.0]undec-7-ene;
DCC for N,N′-dicyclohexylcarbodiimide;
DEAD for diethylazodicarboxylate;
DIAD for diisopropyl azodicarboxylate;
DIPEA or (i-Pr)₂EtN for N,N,-diisopropylethyl amine;
Dess-Martin periodinane for 1,1,1-tris(acetyloxy)-1,1-dihydro-1,2-benziodoxol-3-(1H)-one;
DMAP for 4-dimethylaminopyridine;
DME for 1,2-dimethoxyethane;
DMF for N,N-dimethylformamide;
DMSO for dimethyl sulfoxide;
DPPA for diphenylphosphoryl azide;
EDC for N-(3-dimethylaminopropyl)-N′-ethylcarbodiimide;
EDC HCl for N-(3-dimethylaminopropyl)-N′-ethylcarbodiimide hydrochloride;
EtOAc for ethyl acetate;
EtOH for ethanol;
Et₂O for diethyl ether;
HATU for O-(7-azabenzotriazol-1-yl)-N,N,N′,N′,-tetramethyluronium Hexafluorophosphate;
HCl for hydrogen chloride;
HOBT for 1-hydroxybenzotriazole;
K₂CO₃ for potassium carbonate;
n-BuLi for n-butyl lithium;
i-BuLi for i-butyl lithium;
t-BuLi for t-butyl lithium;
PhLi for phenyl lithium;
LDA for lithium diisopropylamide;
TMEDA for N,N,N′,N′-tetramethylethylenediamine;
LiTMP for lithium 2,2,6,6-tetramethylpiperidinate;
MeOH for methanol;
Mg for magnesium;
MOM for methoxymethyl;
Ms for mesyl or —SO₂—CH₃;
Ms₂O for methanesulfonic anhydride or mesyl-anhydride;
NaN(TMS)₂ for sodium bis(trimethylsilyl)amide;
NaCl for sodium chloride;
NaH for sodium hydride;
NaHCO₃ for sodium bicarbonate or sodium hydrogen carbonate;
Na₂CO₃ sodium carbonate;
NaOH for sodium hydroxide;
Na₂SO₄ for sodium sulfate;
NaHSO₃ for sodium bisulfite or sodium hydrogen sulfite;
Na₂S₂O₃ for sodium thiosulfate;
NH₂NH₂ for hydrazine;
NH₄HCO₃ for ammonium bicarbonate;
NH₄Cl for ammonium chloride;
NMMO for N-methylmorpholine N-oxide;
NaIO₄ for sodium periodate;
Ni for nickel;
OH for hydroxy;
OsO₄ for osmium tetroxide;
TEA or Et₃N for triethylamine;
TFA for trifluoroacetic acid;
THF for tetrahydrofuran;
TPP or PPh₃ for triphenylphosphine;
Troc for 2,2,2-trichloroethyl carbonyl;
Ts for tosyl or —SO₂—C₆H₄CH₃;
Ts₂O for tolylsulfonic anhydride or tosyl-anhydride;
TsOH for p-tolylsulfonic acid;
Pd for palladium;
Ph for phenyl;
POPd for dihydrogen dichlorobis(di-tert-butylphosphinito-κP)palladate (II);
Pd₂(dba)₃ for tris(dibenzylideneacetone)dipalladium (0);
Pd(PPh₃)₄ for tetrakis(triphenylphosphine)palladium (0);
PdCl₂(Ph₃P)₂ for trans-dichlorobis(triphenylphosphine)palladium (II);
Pt for platinum;
Rh for rhodium;
Ru for ruthenium;
TBS for tert-butyl dimethylsilyl; or
TMS for trimethylsilyl;
TMSCl for trimethylsilyl chloride.

### EXAMPLES

Compounds of the invention can be prepared according to U.S. Pat. Nos. 6,753,415; 6,710,034; 7,129,221; 6,878,691; 6,753,318; 6,841,664; 7,049,417; 6,645,941; 6,764,998; 7,276,487; 7,229,972; 7,271,155; and U.S. application Ser. Nos. 11/236,043; 11/828,473 and 11/742,794, which are all incorporated herein by reference.

### Biological Assays

### I. Protocol for In Vitro Inflammatory Assay:

1. Jurkat cell line expressing a luciferase reporter under the control of an NFkB promoter sequence was created. Clones were isolated using G418 and then characterized for enhanced luciferase activity following stimulation of cells with either TNFα or PMA+Ionomycin.
2. Assay protocol was as follows:
   Day 1: Cells were split to a density of 1.1×10⁵ cells/mL.
   Day 2:
      Cells were adjusted to a density of 2×10⁵/mL.
      100 μl of cells were added to each well of a 96-well plate.
      Test compounds were diluted to 200 μg/mL in media (1% DMSO).
      100 μl of diluted test compound was added to cells. Final test compound concentration from 1-120 μL was added and the final DMSO concentration=0.5%. Compounds were tested in duplicate.
      Plates were incubated 1 hour at 37° C.
      10 μl of a 2.1 nM TNFα solution was added to each well. Plates were returned to 37° C. for another 4 hours.
      TNFα-induced luciferase activity was determined using commercially available kits and a microplate luminometer.
      Inhibition of NFkB activity was determined by comparing luciferase activity from test compound-treated cells to luciferase activity in 0.5% DMSO-treated (control) cells.

TABLE 1
In vitro inflammatory data

| Entry | Compound | IC50 (μM) |
|---|---|---|
| 4 | Erythromycin | Inactive |
| 5 | Clarithromycin | >120 (~20% |
| | | inhibition at ~120 |
| 6 | Josamycin | >120 (~20% |
| | | inhibition at ~120 |
| | | μM) |
| 7 | Tylosin | >120 (~20% |
| | | inhibition at ~120 |
| | | μM) |
| 8 | 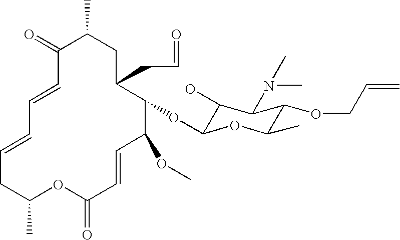 | 41 |
| 9 | 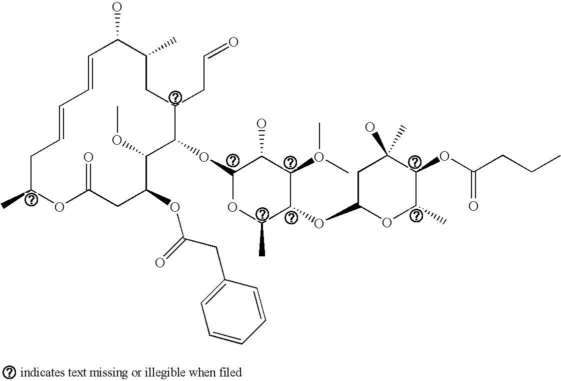 | 31 |
| 10 | 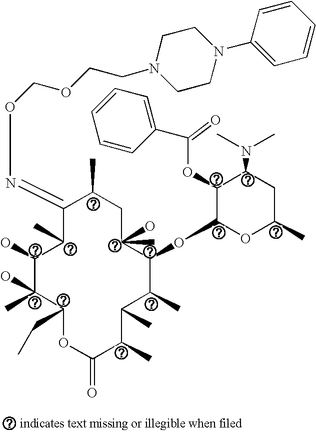 | 30 |
| 11 | 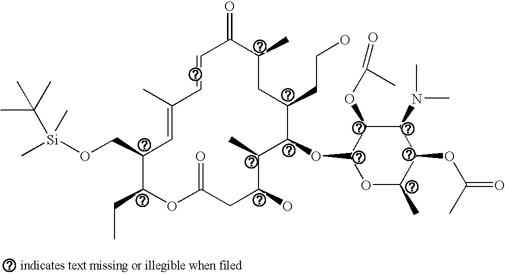 | 22 |
| 12 | 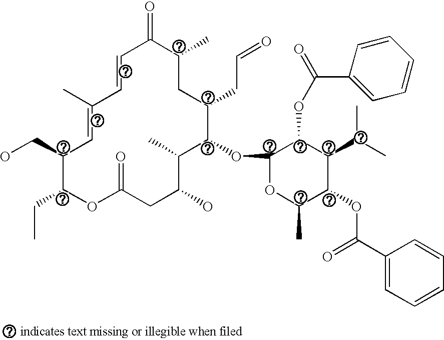 | 17 |

### II. Protocol for In Vivo Rat Dextran Sulfate Sodium (DSS)-Induced Colitis Model:

Rats were treated with a 3% solution of dextrane sulfate sodium (DSS) in water for 11 days to induced colitis. On day 11^{th}, body weight, stool score and blood Hb level were determined. The rats that passed the evaluation on day 11^{th} as having colitis were treated with water for 7 days followed by another body weight evaluation. The rats that were still having inflammation were treated with 3% DSS and test compounds or a positive control compound (sulfasalazine=SASP is used in this experiment) for 7 days (dosage and frequency of dosing dependent on the type of compounds). At the end of 7 days of treatment with tested compounds, the rats were evaluated. Evaluations items include but not limited to lesion area of the colon, changes in body weights, water consumption, stool score, colon full length, plasma haptoglobin, and hematology.

Representative compounds were found to show activity on DSS-induced colitis in rats (i.e. lesions area of colon). In addition compound (3) exhibit improved activity in vitro inflammatory assay when compared with Erythromycin, Clarithromycin, Josamycin and Tylosin.

Although the invention has been described with respect to various preferred embodiments, it is not intended to be limited thereto, but rather those skilled in the art will recognize that variations and modifications may be made therein which are within the spirit of the invention and the scope of the appended claims.

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

What is claimed is:

1. A method of treating inflammatory bowel disease (IBD) using bridged macrolide system represented by formula (I), (II), (III), (IV) or tylosin derivatives of formula (V) as illustrated below: 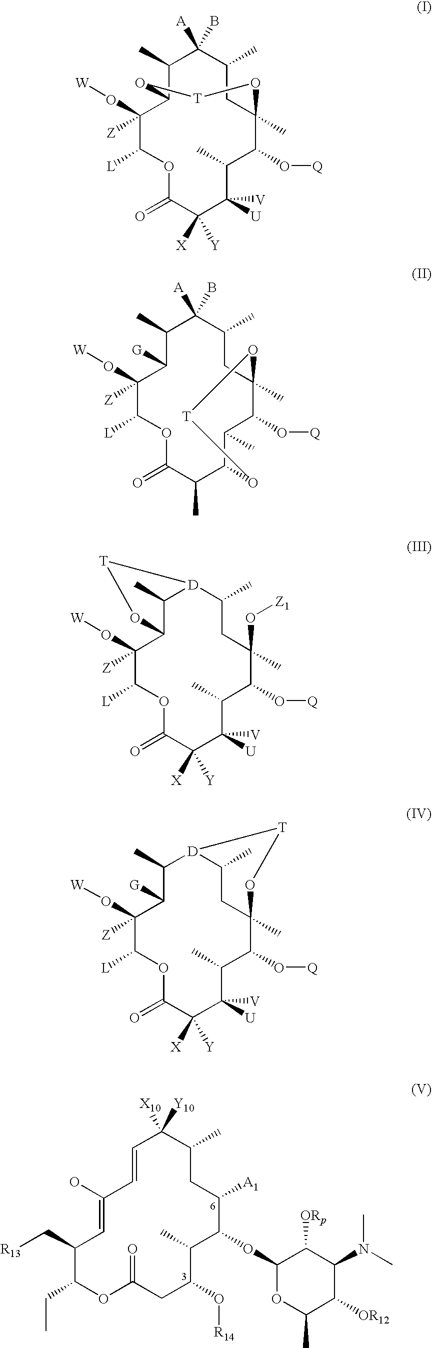
or the racemates, enantiomers, diastereomers, geometric isomers, tautomers, solvates, pharmaceutically acceptable salts, esters and prodrugs thereof,
wherein T is:
(a) —R₁—, where R₁ is substituted or unsubstituted —C₁-C₈ alkylene-, —C₂-C₈ alkenylene- or —C₂-C₈ alkynylene-, containing 0, 1, 2, or 3 heteroatoms selected from O, S or N;
(b) —R₁—(C═O)—R₂—, where R₂ is independently selected from R₁;
(c) —R₁—(C═N-E-R₃)—R₂—, where E is absent, O, NH, NH(CO), NH(CO)NH or NHSO₂ and where R₃ is independently selected from the group consisting of:
(v) hydrogen;
(vi) aryl; substituted aryl; heteroaryl; substituted heteroaryl; and
(vii) —R₄, where R₄ is substituted or unsubstituted —C₁-C₆ alkyl, —C₂-C₆ alkenyl, or —C₂-C₆ alkynyl containing 0, 1, 2, or 3 heteroatoms selected from O, S or N; or
(viii) —R₅, where R₅ is substituted and unsubstituted —C₃-C₁₂ cycloalkyl containing 0, 1, 2, or 3 heteroatoms selected from O, S or N; and
(d) —R₁—[C(OR₆)(OR₇)]—R₂—, where R₆ and R₇ are selected from the group consisting of C₁-C₁₂ alkyl, aryl or substituted aryl; or R₆ and R₇ taken together is —(CRₐR_{b})ᵣ—, where r is 2 or 3, Rₐ and R_{b} are independently selected from R₃;
(e) —R₁—[C(SR₆)(SR₇)]—R₂—; or
(f) —R₁—(C═CH—R₃)—R₂—;
one of A and B is hydrogen or hydroxy and the other is selected from:
(a) hydrogen;
(b) —OR₃;
(c) —R₄;
(d) —OC(O)NHR₃;
(e) —OC(O)OR₃;
(f) —NR₈R₉; where R₈ and R₉ are each independently selected from R₃; alternatively, R₈ and R₉ taken together with the nitrogen atom to which they are connected form a 3- to 10-membered ring which may optionally contain one or more heterofunctions selected from the group consisting of: —O—, —NH—, —N(C₁-C₆-alkyl)-, —N(R₁₀)—, —S(O)ₙ—, wherein n=0, 1 or 2, and R₁₀ is selected from aryl; substituted aryl; heteroaryl; and substituted heteroaryl;
(g) —NHC(O)R₃;
(h) —NHS(O)₂R₃;
(i) —NHC(O)OR₃; and
(j) —NHC(O)NHR₃;
alternatively, A and B taken together with the carbon atom to which they are attached are selected from:
(a) C═O;
(b) C═N-J-R₁₁, where J is absent, O, C(O), SO₂, NH, NHC(O), NHC(O)NH or NHSO₂; and wherein R₁₁ is independently selected from halogen and R₃;
(c) C═CH-J-R₁₁;
(d) substituted or unsubstituted, and saturated or unsaturated 5- to 10-membered heterocyclic;
D is 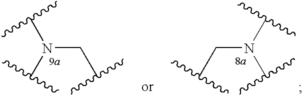
G is selected from the group consisting of:
a) hydrogen;
b) hydroxy;
c) —O—R₄;
d) —O—R₁₀;
Alternatively, G and W taken together to a form cyclic structure selected from:
a) 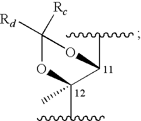
where R_{c} and R_{d} are independent selected from R₃, and
b) 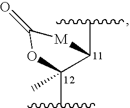
where M is O or N-J-R₂₀, and where J is absent, O, NH, NHC(O), or N═CH; and R₂₀ is selected from the group consisting of:
i. hydrogen;
ii. R₄; and
iii. R₁₀;
W is selected from:
(a) hydrogen;
(b) —R₄;
(c) —C(O)R₃;
(d) —C(O)O—R₃; and
(e) —C(O)N(R₈R₉);
when U is hydrogen, V is selected from the group consisting of:
(a) hydrogen;
(b) —OR₃;
(c) —OC(O)R₃;
(d) —OC(O)NHR₃;
(e) —OS(O)₂R₃;
(f) —O-monosaccharide; and
(g) —O-disaccharide;
alternatively, U and V taken together is oxo;
L is independently selected from R₄;
Q is:
(a) —R₃;
(b) —C(O)R₃;
(c) —C(O)NHR₃;
(d) —C(O)OR₃;
(e) —S(O)₂R₃;
(f) monosaccharide;
(g) disaccharide; or
(h) trisaccharide;
Z is:
(a) hydrogen;
(b) —N₃;
(c) —CN;
(d) —NO₂;
(e) —CONH₂;
(f) —COOH;
(g) —CHO;
(h) —R₄;
(i) —COOR₄;
(j) —C(O)R₄; or
(k) —C(O)NR₈R₉;
Z₁ is hydrogen or —R₄;
Each of X and Y is independently:
a) hydrogen;
b) hydroxy;
c) halogen; or
d) —R₄;
A₁ is selected from the group consisting of:
a) CH₂CHO;
b) CH₂CN;
c) CH₂C═N-J-R₁₁;
d) CH₂E₁R₃₀, where E₁ is absent, O, OC(O), C(O), C(O)NR₃₁, SO₂, CH₂, NR₃₁, R₁, NR₃₁C(O)NR₃₁, NR₃₁SO₂NR₃₂ or NR₃₁SO₂; and wherein R₃₀, R₃₁ and R₃₃ are independently selected from halogen and R₃;
R₁₄ is selected from the group consisting of:
a) hydrogen;
b) hydroxy protecting group;
c) hydroxy prodrug group;
d) —R₄;
e) —C(O)R₃;
f) —C(O)O—R₃; and
g) —C(O)N(R₈R₉);
alternatively, A₁ and R₁₄ can be taken together with the atoms to which they are attached to form 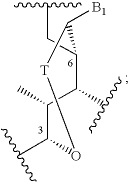
where B₁ is selected from the group consisting of:
a) CHO;
b) CN;
c) HC═N-J-R₁₁;
d) J₁R₃₀, where J₁ is absent, O, OC(O), SO₂, CH₂, NR₃₁, R₁, NR₃₁C(O)NR₃₁, NR₃₁NR₃₂, NR₃₁NR₃₂C(O), NR₃₁SO₂NR₃₂ or NR₃₁SO₂; and wherein R₃₀, R₃₁ and R₃₂ are independently selected from halogen and R₃;
X₁₀ and Y₁₀ are each independently selected from the group consisting of:
(a) hydrogen;
(b) halogen;
(c) protected hydroxy;
(d) -E-R₃; and
(e) —NR₇R₈;
Alternatively, X₁₀ and Y₁₀ taken together with the carbon atom to which they are attached is:
(a) C═O;
(b) C═N—C(O)R₃;
(c) C═N—OR₉₀, wherein R₉₀ is selected from the group consisting of:
(1) hydrogen;
(2) —CH₂O(CH₂)₂OCH₃;
(3) —CH₂O(CH₂O)ₙCH₃, wherein n is 1, 2, or 3;
(4) —R₄;
(5) substituted and unsubstituted, saturated or unsaturated C₃-C₁₂ cycloalkyl;
(6) substituted and unsubstituted heterocyclic;
(7) C(O)—(C₃-C₁₂ cycloalkyl);
(8) C(O)—R₃, wherein R₃ is as previously defined;
(9) —Si(Rₐ)(R_{b})(R_{c}), wherein Rₐ, R_{b} and R_{c} are each independently selected from the group consisting of C₁-C₁₂ alkyl, aryl and substituted aryl; or
(10) C(R₉₀)(R₁₀₀)—O—R₁₁₀, wherein R₉₀ and R₁₀₀ taken together with the carbon atom to which they are attached form a C₃ to C₁₂ cycloalkyl group or each independently is selected from the group consisting of: hydrogen and C₁-C₁₂ alkyl; and R₁₁₀ is selected from the group consisting of:
(i) —R₄;
(ii) substituted and unsubstituted, saturated or unsaturated —C₃-C₁₂ cycloalkyl; and
(iii) —Si(Rₐ)(R_{b})(R_{c}), wherein Rₐ, R_{b} and R_{c} are as previously defined;
R₁₂ is -M₁-Q₁,
where M₁ is:
(a) absent;
(b) —C(O)—;
(c) —C(O)N(R₃)—; or
(d) —R₁—;
and where Q₁ is:
(a) hydrogen;
(b) hydroxy protecting group;
(c) hydroxy prodrug group;
(d) 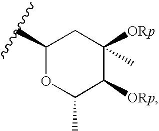
where Rp is hydrogen, a hydroxy protecting group or a hydroxy prodrug group;
(e) —R₃;
(f) —OR₃;
(g) —NR₇R₈; or
(h) substituted or unsubstituted heterocyclic;
R₁₃ is -G₁-M₁-W₁, where G₁ is absent, —O—, or —N(R₃)—, and where W₁ is:
(a) hydrogen;
(b) hydroxy protecting group;
(c) hydroxy prodrug group;
(d) halogen;
(e) 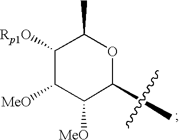
(e)
(f) —R₃;
(g) —OR₃; or
(h) substituted or unsubstituted heterocyclic;
Rₚ and Rₚ₁ are independently hydrogen, a hydroxy protecting group or a hydroxy prodrug group.

2. The method according to claim 1 with a compound represented by formula (VI), or a pharmaceutically acceptable salt, ester or prodrug thereof: 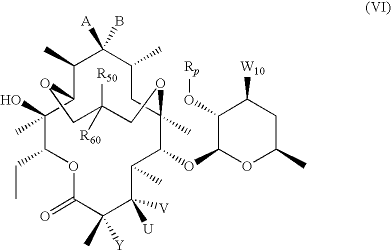
where R₅₀ and R₆₀ are independently selected from the group consisting of:
a) hydrogen;
b) deuterium;
c) hydroxy;
d) activated hydroxy;
e) N₃;
f) NH₂;
g) CN;
h) protected hydroxy;
i) protected amino;
j) -L₁-R₃, where L₁ is absent, O, OC(O), S, S(O), SO₂, NH, NCH₃, NHC(O), NHC(O)NH or NHSO₂; and
k) substituted or unsubstituted heterocyclic;
alternatively, R₅₀ and R₆₀ can be taken together with the carbon atom to which they are attached is selected from the group consisting of:
a) C═O;
b) C(OR₆)(OR₇);
c) C(SR₆)(SR₇);
d) C═CHR₃;
e) C═NRₐₚ; where Rₐₚ is amino protecting group; and
f) C═N-E-R₃;
W₁₀ is —NR₈R₉; and A, B, U, V, Y, R₃, R₆, R₇, R₈, R₉ and Rₚ are as previously defined in claim 1.

3. The method according to claim 1 with a compound represented by formula (VII), or a pharmaceutically acceptable salt, ester or prodrug thereof: 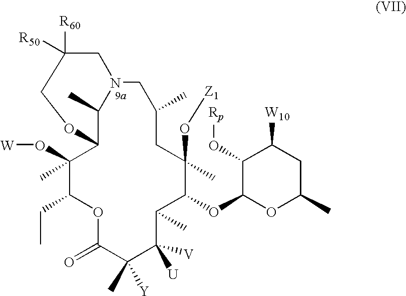
where R₅₀, R₆₀, U, V, Y, W, W₁₀, Z₁ and Rₚ are as previously defined in claims 1 and 2.

4. The method according to claim 1 with a compound represented by formula (VIII), or a pharmaceutically acceptable salt, ester or prodrug thereof: 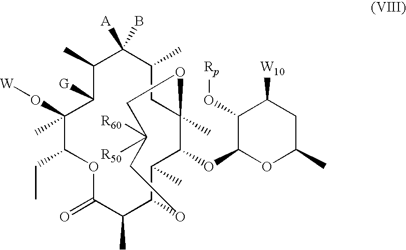
where R₅₀, R₆₀, A, B, G, W, W₁₀ and Rₚ are as previously defined in claims 1 and 2.

5. The method according to claim 1 with a compound represented by formula (IX), or a pharmaceutically acceptable salt, ester or prodrug thereof: 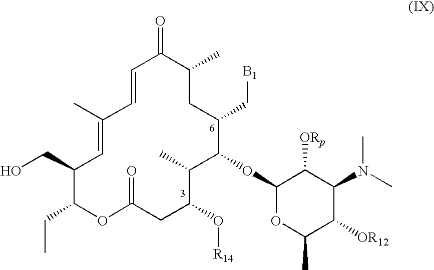
where B₁, R₁₂, R₁₄ and Rₚ are as previously defined in claim 1.

6. The method according to claim 1 with a compound select from the group consisting of:

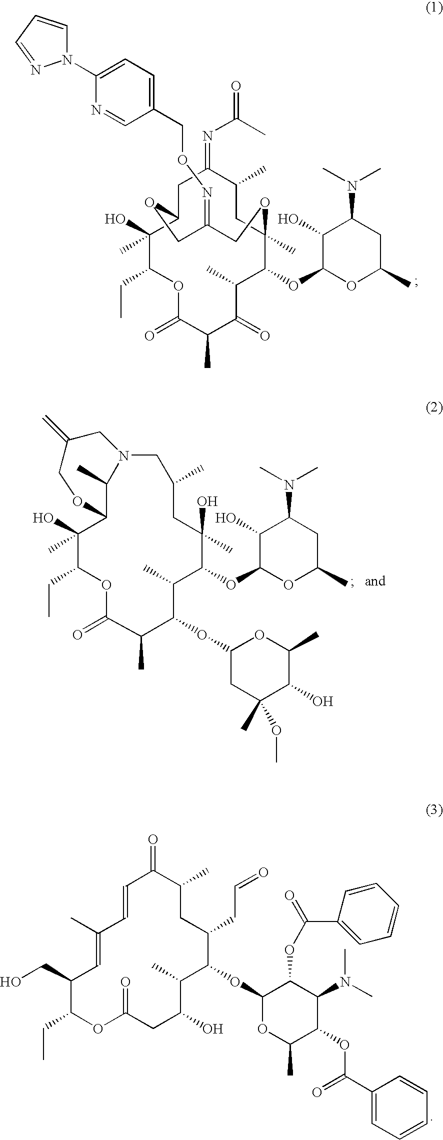

7. The method according to claim 1, wherein IBD is Crohn's disease or ulcerative colitis.

8. The method according to claim 1 further comprising one or more antibiotics or a pharmaceutically acceptable salt, ester, or prodrug thereof.

9. The method according to claim 1 further comprising one or more drugs used in the treatment of IBD.

10. The method according to claim 9, wherein the drug is selected from the group consisting of auranofin, azathioprine, cyclophosphamide, cyclosporine, etanercept, hydroxychloroquine, inflaximab, leflunomide, methotrexate, minocycline, mycophenalate mofetil, penicillamine, sulfasalazine, tacrolimus, and corticosteroids.

11. A method of treating symptoms of IBD selected from abdominal pain, diarrhea, rectal bleeding, weight loss, fever, loss of appetite, and other more serious complications, such as dehydration, anemia and malnutrition using a compound of formula (I), (II), (III), (IV), (V), or combination thereof.
